(19) United States
(12) Patent Application Publication
(10) Pub. No.: US 2017/0369831 A1
(43) Pub. Date: Dec. 28, 2017

(54) SYSTEM AND PROCESS FOR ENHANCING SENSORY ATTRIBUTES IN A BEVERAGE

(71) Applicant: E. & J. Gallo Winery, Modesto, CA (US)

(72) Inventors: Ajwinder Dhesi, Modesto, CA (US); Rachel Dokich, Modesto, CA (US); Sameen Kiayei, Modesto, CA (US); Raley Lee, Modesto, CA (US); Dharma Bangalore, Modesto, CA (US); Dhruba Das, Modesto, CA (US); David Warter, Modesto, CA (US); Ravi Ponangi, Modesto, CA (US); Richard Ntuli, Modesto, CA (US)

(73) Assignee: E.& J. Gallo Winery, Modesto, CA (US)

(21) Appl. No.: 15/632,194

(22) Filed: Jun. 23, 2017

Related U.S. Application Data
(60) Provisional application No. 62/354,052, filed on Jun. 23, 2016.

(51) Int. Cl. C12M 1/42; C12N 13/00; C12H 1/16

(52) U.S. Cl. CPC C12M 35/04; C12H 1/16; C12N 13/00

(57) ABSTRACT

A system and process for enhancing sensory attributes of a beverage product are described. According to one embodiment, the process comprises providing a consumable beverage. A sonication process is applied to the consumable beverage utilizing a sonication unit. Chemical reactions in the consumable beverage are catalyzed to modify a sensory attribute of the consumable beverage.

### CROSS REFERENCE TO RELATED APPLICATIONS

This application claims priority U.S. provisional patent application No. 62/354,052 filed Jun. 23, 2016 entitled, “System and Process for Enhancing Sensory Attributes in a Beverage,” which is hereby incorporated by reference in its entirety.

### FIELD OF THE INVENTION

The present invention is generally directed to processes for maturing beverage products. Specifically, the present invention is directed to processes for enhancing sensory attributes in an alcoholic beverage.

### BACKGROUND OF THE INVENTION

Alcoholic beverages or drinks are generally grouped into two categories of beverages including distilled drinks and fermented drinks. Distilled drinks include alcoholic beverages such as whiskey, vodka, rum and tequila. Fermented drinks include alcoholic beverages such as beer, wine, cider and mead. Distilled drinks are produced by a process known as distillation, whereby ethanol, which is produced by fermenting grains, fruits, and vegetables, is distilled. The distillation of fermented products produces distilled drinks with high alcohol content. A fermented drink, such as beer, may be produced by a process of brewing, which involves the fermentation of sugars.

Techniques may be employed to improve the sensory attributes of an alcoholic beverage after it is produced, such as attributes affecting taste, color, aroma, and smoothness of the beverages. Beverage maturing is a widely known and used technique for improving the sensory attributes of an alcoholic beverage. For example, wine and distilled beverages are commonly aged to enhance the taste and smoothness of these drinks. An aging barrel may be used to mature an alcoholic beverage. Aging barrels are commonly made of wood, such as an oak, redwood or chestnut. Alcoholic beverages aged in wooden barrels take on some of the compounds of the barrel, such as tannins. Tannins, for example, have been known to significantly contribute to the aroma and taste of distilled drinks and non-distilled drinks.

The process of maturing, however, involves procuring flavor by causing the alcohol beverage to stand for months or years in a wooden ageing barrel while allowing organic material in the wood to be extracted. During the aging process, tannins are just one component of the wood that is extracted over a period of years. However, rapid aging techniques, such as rapid tannin extraction, have been developed to accelerate the maturation of alcoholic beverages. Still, these techniques have not been successful in replicating the characteristics associated with a mature flavor accomplished by traditional methods relying on environmental conditions and the chemical reactions that occur over time.

### SUMMARY

Briefly, and in general terms, various embodiments are directed to a system and method for enhancing sensory attributes of a beverage product. A system and process for enhancing sensory attributes of a beverage product are described. According to one embodiment, the process comprises providing a consumable beverage. A sonication process is applied to the consumable beverage utilizing a sonication unit. Chemical reactions in the consumable beverage are catalyzed to modify a sensory attribute of the consumable beverage.

Other features and advantages will become apparent from the following detailed description, taken in conjunction with the accompanying drawings, which illustrate by way of example, the features of the various embodiments.

### BRIEF DESCRIPTION OF THE DRAWING

The accompanying drawings, which are included as part of the present specification, illustrate the various embodiments of the present disclosed system and method and together with the general description given above and the detailed description of the preferred embodiment given below serve to explain and teach the principles of the present disclosure.

FIG. 1 depicts an example beverage processing system for enhancing a sensory attribute of a beverage, according to an exemplary embodiment of the present disclosure.

FIG. 2 is flow chart that illustrates a process of enhancing a sensory attribute of a beverage, according to an exemplary embodiment of the present disclosure.

It should be noted that the figures are not necessarily drawn to scale and that elements of structures or functions are generally represented by reference numerals for illustrative purposes throughout the figures. Perforated lines in the Figures denote components or steps that may be optional in one or more embodiments of the present disclosure. It also should be noted that the figures are only intended to facilitate the description of the various embodiments described herein. The figures do not describe every aspect of the teachings described herein and do not limit the scope of the claims.

### DETAILED DESCRIPTION

The present disclosure provides an improvement over existing systems and processes for maturing beverages. By way of example only, the disclosed technology may be used for enhancing a sensory attribute in a beverage. The present disclosure is related to a sonication process for enhancing sensory attributes in a beverage. Energy induced by the sonication process of the present disclosure catalyzes chemical reactions and produces beverage products with enhanced sensory attributes. According to one embodiment, this is accomplished using High Energy Short Time (HEST). According to an exemplary embodiment, the beverage may be whiskey. The disclosed technology, however, may be used for any type of alcoholic beverage.

For purposes of the present disclosure, a sensory attribute may be a chemical or physical property of a beverage that has an effect on the human senses, including but not limited to chemical or physical properties affecting an aroma, color, flavor, texture and/or smoothness of a beverage.

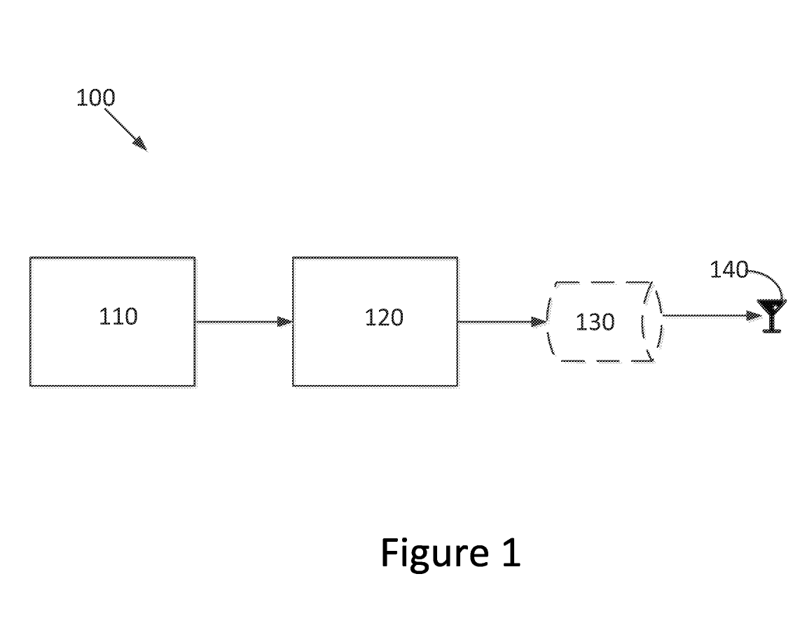

Referring now to FIG. 1, one exemplary beverage processing system 100 for enhancing a sensory attribute of a beverage 110 is shown. In particular, FIG. 1 illustrates a beverage base 110, a sonication unit 120, a barrel 130, and a beverage product 140.

As depicted in FIG. 1, the beverage base 110 is provided to the sonication unit 120 in order to process the beverage base 110 and produce the beverage product 140. In an exemplary embodiment, beverage base 110 is a consumable alcoholic beverage or drink. According to one embodiment, the beverage base 110 may be a type of distilled alcoholic beverage, such as whiskey or liquor. According to another embodiment, the beverage base 110 may be a type of fermented alcoholic beverage, such as beer, wine, mead, or cider. According to yet another embodiment, beverage base 110 may be any alcoholic beverage with an alcohol content ranging from 10 to 193.6 proof.

In an exemplary embodiment, the sonication unit 120 may be a liquid processor capable of receiving a beverage base 110 and subjecting the beverage base 110 to a process of sonication. According to one embodiment, sonication may be a process of applying sound energy to agitate particles in a sample, e.g. sonicating the sample to produce an effect on the chemical and/or physical properties of the sample. In one embodiment, sonication refers to sonicating the beverage base 110, e.g. whiskey, with an ultrasonic processor unit at set conditions which enable modification to the attributes of the beverage base 110. According to one embodiment, the sonication unit 120 may be an ultrasonic processor unit capable of using ultrasonic frequencies, i.e. frequencies above 20 KHz. In this embodiment, sonication using ultrasonic frequencies leads to the process of ultra-sonication. In one embodiment, the sonication unit 120 may sonicate at an ultrasonic frequency inducing energy at a power of 1000-2000 W/L. As an illustration, the sonication unit 120 may include an ultrasonic processor which delivers 400 watts of power at a frequency of 24 KHz.

In one embodiment, the sonication unit 120 may be any of several commercial ultrasonic devices for the ultra-sonication of liquids in different volumes, such as an ultrasonic device made by Hielscher™, for lab samples, pilot scale processing or full scale processing. For example, the sonication unit 120 may be a compact handheld device capable of holding up to 500 mL, a laboratory device capable of receiving up to 2000 mL, or an industrial ultrasonic processor used for larger volume processing. In one embodiment, the sonication unit 120, operating under set conditions, may be used to sonicate a sample of whiskey to enable modification to sensory attributes of the whiskey sample and produce an enhanced or improved tasting and/or smelling beverage product 140.

In one embodiment, the beverage processing system 100 may include an additional device, appliance, or apparatus which may be used to further enhance or improve the beverage product 110. In one embodiment, as shown in FIG. 1, the barrel 130 may be used for aging the beverage after it has been processed by the sonication unit 120 to produce the beverage product 140. The container 130 may be a cask or a tun. In one embodiment, the barrel 130 is a hollow cylindrical container made of wooden staves and bounded by wooden or metal hoops. In one embodiment, the wooden staves of barrel 130 are made of oak or maple. In one embodiment, the barrel 130 may be used as a vessel to treat the sonicated beverage 110 with additives and flavorants such as oak or other carbon sources. Additives and flavorants may be used to enhance the aroma, color, flavor, and/or smoothness of a beverage, such as beverage 110. Examples of additives and flavorants that may be used for enhancing a beverage, such as beverage 110, include woods, plant extracts, vegetables, seeds, nuts, and fruits.

In an exemplary embodiment, the beverage product 140 is an enhanced beverage base 110 that has undergone a sonication process at ultrasonic frequencies, such as frequencies above 24 KHz. In one embodiment, the beverage product 140 is an enhanced consumable alcoholic beverage or drink. In one embodiment, the beverage product 140 may have been produced from a process of aging in barrel 130 after being processed by sonication unit 120. In one embodiment, the beverage product 140 may have been treated with or contacted with additives and flavorants before, after, or at the same time as being processed by sonication unit 120.

According to one embodiment of the present system 100, including the sonication unit 120, may have the following characteristics:
an ultrasonic processor, (e.g., the UP 400S made by Hielscher Ultrasonics Gmbh™) delivering 400 Watts @ 24 KHz,
the beverage is 80-120 proof,
40% Amplitude,
5 min sonication,
full-cycle: (e.g. A 100% wave pulse introduced into the base 110),
energy is input at 84 kilowatts/liter, and
temperature of the beverage during processing ranges from 60° F. to 140° F.

Additional combinations of characteristics, ranges, and settings of the sonication unit 120 are within the scope of the present disclosure.

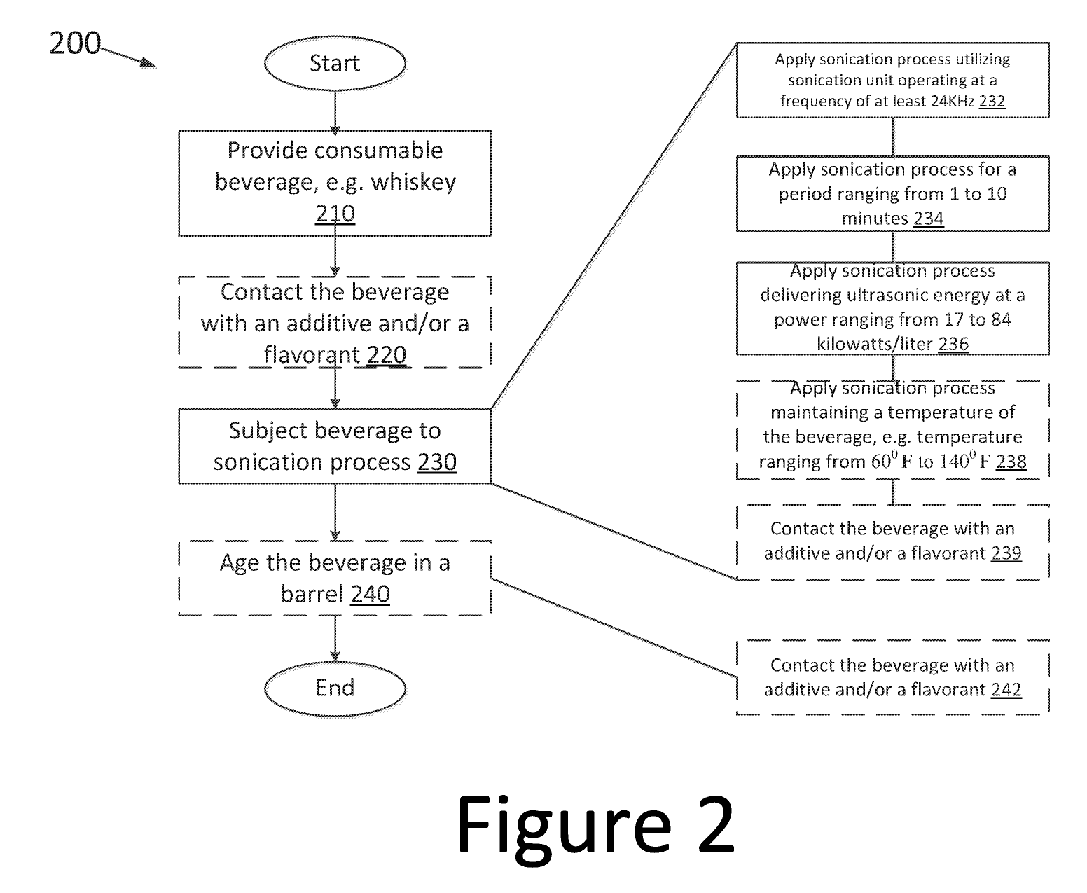

Referring now to FIG. 2, this figure illustrates an exemplary process 200 of enhancing a sensory attribute of a beverage by system 100. At the start of the process, a consumable beverage, such as beverage base 110, may be provided at 210. In one embodiment, the consumable beverage may be provided in different volumes depending on the quantity specifications of the sonication unit 120 that will used to perform a sonication process. In one embodiment, at 210, the consumable beverage may be provided directly to a sonication unit 120 for sonication of the consumable beverage 110. In one embodiment, at 210, the consumable beverage 110 may be provided to an apparatus such as an aging barrel for treating the beverage 110 prior to sonication. In one embodiment, an aging barrel 130 may be used for contacting, at 220, the beverage base 110 with one or more additives or flavorants such as oak or other carbon sources.

In an exemplary embodiment of the present disclosure, the provided beverage base 110 is subjected, at 230, to sonication (e.g., an ultrasound) in order to enhance the sensory attributes of the consumable beverage base 110. For example, the sonication process, at 230, may be performed on a consumable base of 80 proof straight bourbon whiskey containing 50% alcohol by volume (ABV) using the sonication unit 120. As illustrated in FIG. 2, a number of conditions or settings of the sonication unit 120, including an ultrasonic frequency, may be employed and maintained to facilitate the sonication process, at 230. In an exemplary embodiment of the present disclosure, the sonication process requires that the sonication unit 120 operates, at 232, at a frequency of 24 KHz or higher. In one embodiment, the sonication process is applied, at step 234, on the beverage base 110 for a period of time ranging from 1 to 10 minutes at a predetermined frequency. For example, the sonication process may proceed from 1 to 5 minutes at a frequency of 24 KHz or higher. In this example, the period of time may be extended up to, and not to exceed, 10 minutes.

According to an exemplary embodiment of the present disclosure, energy may be induced by the sonication process 230 to catalyze or accelerate chemical reactions in the beverage 110 and produce a beverage product 140 with enhanced sensory attributes. In one embodiment, the beverage base 110 is subjected, at 230, to sonication delivering, at 236, ultrasonic energy at a power ranging from 17 to 84 kilowatts/liter. In one embodiment, ultrasonic energy is delivered via the sonication unit operating in the frequency range from 24 KHz to 270 KHz. In yet a further embodiment, the sonication unit may deliver energy in the megasonic range from 350 KHz to 1.3 MHz. In the embodiments described above, additional combinations or processing characteristics are within the scope of the present disclosure.

In one embodiment, the process of enhancing the sensory attributes of beverage base 110 may involve maintaining, at 238, a temperature of the beverage 110 while the beverage is subjected to ultrasonic energy during the sonication process 230. For example, the temperature of the beverage base 110 may be maintained at temperatures ranging from 60° F. to 140° F. while the beverage is being subjected to ultrasonic energy during the sonication process 230. In one embodiment, the beverage base 110 may be contacted, at 239, with one or more additives and/or flavorants while both ingredients are being subjected to ultrasonic energy during the sonication process 230. It is within the scope of the present disclosure that additives and flavorants may be contacted with either the processed or unprocessed beverage 110 at any stage of the process 200, including before, after, or at the same time as being subjected to the sonication process at 230. For example, following sonication at 230, the process 200 may include a step of aging the processed beverage, i.e. the beverage 110 that has undergone sonication at 230, in the barrel 130 to yield the final beverage product 140. In one embodiment, the processed beverage may be treated, at 242, with additives or flavorants while aging, at 240, the processed beverage in a wooden barrel, as described above. For example, process 200 may conclude with treating the processed beverage with carbon and the use of oak chips to yield the beverage product 140 that has enhanced sensory attributes.

The above example embodiments have been described herein to illustrate various embodiments of a system 100 and a process 200 for enhancing sensory attributes of a beverage. If used on whiskeys, the process 200 using the system 100 may cause an increase in grain (rye) and spice attributes and a decrease in undesirable resin notes of a finished whiskey product. Further, relatively smaller changes to sensory attributes in terms of fruit, heat, sweet aromatics, toast attributes and mouthfeel may be observed. Various modifications and departures from the disclosed example embodiments will occur to those having ordinary skill in the art.

One of ordinary skill in the art will appreciate that not all systems for producing beverages have all these components and may have other components in addition to, or in lieu of, some components mentioned here. Furthermore, while these components are viewed and described separately, various components may be integrated into a single unit in some embodiments.

The various embodiments described above are provided by way of illustration only and should not be construed to limit the claimed invention. Numerous alternative embodiments could be implemented, using either current technology or technology developed after the filing date of this application. Those skilled in the art will readily recognize various modifications and changes that may be made to the claimed invention without following the example embodiments and applications illustrated and described herein, and without departing from the true spirit and scope of the claimed invention, which is set forth in the following claims.

What is claimed:

1. A process, comprising:
providing a consumable beverage;
applying a sonication process to the consumable beverage utilizing a sonication unit, and
catalyzing chemical reactions in the consumable beverage to modify a sensory attribute of the consumable beverage.

2. The process of claim 1, wherein the consumable beverage is a whiskey.

3. The process of claim 1, wherein the consumable beverage is a beverage with alcohol content ranging from 10 to 193.6 proof.

4. The process of claim 1, wherein the sonication process is applied to the beverage for a period of time ranging from one (1) to five (5) minutes.

5. The process of claim 1, further comprising maintaining a temperature of the beverage ranging from 60° F. to 140° F. during the sonication process.

6. The process of claim 1, wherein the modified sensory attributes of the whiskey include one or more of an increase in a grain attribute, an increase in a rye attribute, an increase in a spice attribute, and a decrease in a resin note of the whiskey.

7. The process of claim 1, wherein the sonication unit includes an ultrasonic liquid processor.

8. The process of claim 1, further comprising aging the beverage in a barrel.

9. The process of claim 2, further comprising contacting the beverage with at least one of an additive and a flavorant from an oak or carbon source.

10. A system, comprising:
a sonication unit set to operate at a frequency of at least 24 KHz, the sonication unit configured to perform a sonication process on a consumable beverage base, the sonication unit set to conditions that enable modification of sensory attributes of the beverage via the sonication process, the sonication unit set to apply a sonication process to the beverage for a period of time ranging from 1 to 10 minutes and deliver ultrasonic energy to the beverage at a power ranging from 17 to 84 kilowatts/liter, the ultrasonic energy capable of catalyzing chemical reactions in the beverage to modify a sensory attribute of the beverage.

11. The system of claim 10, wherein the consumable beverage base is a type of whiskey.

12. The system of claim 10, wherein the consumable beverage is a beverage with alcohol content ranging from 10 to 193.6 proof.

13. The system of claim 10, wherein the period of time ranges from one (1) to five (5) minutes.

14. The system of claim 10, the sonication unit is set to maintain a temperature of the beverage ranging from 60° F. to 140° F. during the sonication process.

15. The system of claim 11, wherein the modified sensory attributes of the whiskey include one or more of an increase in a grain attribute, an increase in a rye attribute, an increase in a spice attribute, and a decrease in a resin note of the whiskey.

16. The system of claim 10, wherein the sonication unit includes an ultrasonic liquid processor.

17. The system of claim 10, further including a wooden barrel for aging the beverage.

18. The system of claim 17, wherein the wooden barrel contains at least one of an additive and a flavorant from an oak or carbon source for treating the beverage.

19. A beverage product, the beverage product produced from a process for enhancing a sensory attribute of a beverage base comprising:
providing a consumable beverage base;
applying a sonication process to the consumable beverage base utilizing a sonication unit operating at a frequency of at least 24 KHz, the sonication process being applied to the beverage base for a period of time ranging from 1 to 10 minutes and delivering ultrasonic energy at a power ranging from 17 to 84 kilowatts/liter, the ultrasonic energy catalyzing chemical reactions in the beverage base to modify a sensory attribute of the beverage base.

20. The beverage product of claim 19, wherein the consumable beverage base is a type of whiskey.

21. The beverage product of claim 20, wherein the modified sensory attributes of the whiskey include one or more of an increase in a grain attribute, an increase in a rye attribute, an increase in a spice attribute, and a decrease in a resin note of the whiskey.
